(19) United States
(12) Patent Application Publication
(10) Pub. No.: US 2025/0005964 A1
(43) Pub. Date: Jan. 2, 2025

(54) PREDICTION APPARATUS, PREDICTION METHOD, AND PROGRAM

(71) Applicant: Nippon Telegraph and Telephone Corporation, Tokyo (JP)

(72) Inventors: Daiki SATO, Musashino-shi, Tokyo (JP); Atsushi FUKAYAMA, Musashino-shi, Tokyo (JP); Jiro Nagao, Musashino-shi, Tokyo (JP); Narimune MATSUMURA, Musashino-shi, Tokyo (JP)

(21) Appl. No.: 18/696,457

(22) PCT Filed: Oct. 6, 2021
(86) PCT No.: PCT/JP2021/036931

§ 371 Date: Mar. 28, 2024

(51) Int. Cl. G06V 40/20; G06T 7/73

(52) U.S. Cl. CPC G06V 40/20; G06T 7/74; G06T 2207/10016; G06T 2207/10024; G06T 2207/10028; G06T 2207/20076; G06T 2207/30196

(57) ABSTRACT

A prediction device according to an aspect of the present invention includes: a position information generation part which generates position information of a subject; a degree-of-roaming calculation part which calculates a degree of roaming indicating a degree of stereotypic behavior relating to walking for the subject on the basis of the position information of the subject; a prediction part which predicts occurrence of a predetermined symptom in the subject on the basis of the degree of roaming of the subject; and a notification part which outputs a notification in response to prediction that symptom will occur in the subject.

### TECHNICAL FIELD

The present invention relates to a technique for predicting the occurrence of symptoms such as wandering.

### BACKGROUND ART

Behavioral and psychological symptoms of dementia (BPSD) such as wandering and sleep disorders may appear in people with dementia in some cases. For example, when a symptom of wandering occurs in a person with dementia, an unforeseen situation such as the patient going missing may occur in some cases. In order to prevent the occurrence of unforeseen circumstances, it is important to be able to predict the occurrence of BPSD in people with dementia. For example, NPL 1 discloses a method of detecting the occurrence of wandering by using image analysis using surveillance cameras, radio frequency identification (RFID), Bluetooth (registered trademark) low energy (BLE) beacons, and the like.

### CITATION LIST

### Non Patent Literature

[NPL 1] Daisuke ITO, “Measures for Elderly People with Dementia using IoT for Aging Society”, [online], Nov. 22, 2019, [retrieved Sep. 10, 2021], Internet <URL:https://www.nttpc.co.jp/technology/longevity.html>

### SUMMARY OF INVENTION

### Technical Problem

An object of the present invention is to provide a technique capable of predicting the occurrence of symptoms such as wandering.

### Solution to Problem

A prediction device according to an aspect of the present invention includes: a position information generation part which generates position information of a subject; a degree-of-roaming calculation part which calculates a degree of roaming indicating a degree of stereotypic behavior relating to walking for the subject on the basis of the position information of the subject; a prediction part which predicts occurrence of a predetermined symptom in the subject on the basis of the degree of roaming of the subject; and a notification part which outputs a notification in response to a prediction that a symptom will occur in the subject.

### Advantageous Effects of Invention

According to the present invention, a technique which can predict the occurrence of symptoms such as wandering can be provided.

### BRIEF DESCRIPTION OF DRAWINGS

FIG. 1 is a block diagram showing a prediction system including a prediction device according to an embodiment.

FIG. 2 is a diagram for explaining degree-of-roaming calculation according to the embodiment.

FIG. 3 is a block diagram showing a hardware configuration of the prediction device according to the embodiment.

FIG. 4 is a flowchart for describing a prediction method according to the embodiment.

### DESCRIPTION OF EMBODIMENTS

Embodiments of the present invention will be described below with reference to the drawings.

### [Configuration]

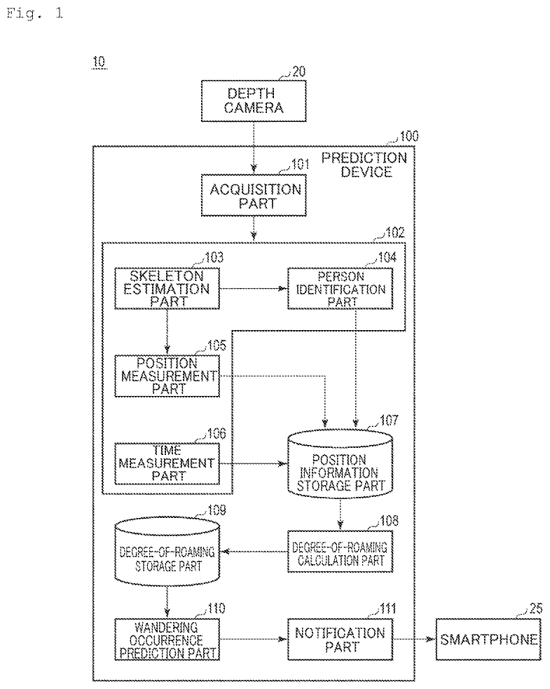

FIG. 1 schematically shows a prediction system 10 including a prediction device 100 according to an embodiment of the present invention. The prediction system 10 is a system for watching over a person with dementia and the prediction device 100 predicts the occurrence of wandering in the person with dementia. Wandering is one of the behavioral and psychological symptoms of dementia (BPSD). Hereinafter, the person with dementia to be watched over is called a subject. Note that a person with dementia is a non-limiting example of a subject.

In the example shown in FIG. 1, the prediction system 10 includes a depth camera 20 and a smartphone 25, in addition to a prediction device 100. The prediction device 100 may be a computer such as a server. The prediction device 100 is communicatively connected to the depth camera 20 and the smartphone 25. The prediction device 100 may communicate with the depth camera 20 and the smartphone 25 over a network such as the Internet.

The depth camera 20 generates RGBD data including a depth image representing depth information of the target space and a color image of the target space and transmits the RGBD data to the prediction device 100. As an example, the depth camera 20 includes a depth sensor which generates depth images and an RGB camera which generates color images. The depth sensor may measure depth information on the basis of, for example, time of flight (ToF) or stereo vision. The depth camera 20 periodically generates pairs of depth and color images and transmits them in real time. For example, the depth camera 20 may generate five image pairs per second.

Typically, the depth camera 20 is placed inside the building in which the subject lives (for example, a residence or a hospital). The target space is, for example, the bedroom of the subject. Although one depth camera 20 is shown in the example shown in FIG. 1, a plurality of depth cameras 20 may be distributed to obtain a wide observation range. Some depth cameras 20 may be placed inside the building and some depth cameras 20 outside the building.

The prediction device 100 includes an acquisition part 101, a position information generation part 102, a position information storage part 107, a degree-of-roaming calculation part 108, a degree-of-roaming storage part 109, a wandering occurrence prediction part 110, and a notification part 111.

The acquisition part 101 acquires RGBD data from the depth camera 20. The RGBD data includes depth images and color images.

The position information generation part 102 generates position information indicating the position of the subject on the basis of the RGBD data acquired using the acquisition part 101. In the example shown in FIG. 1, the position information generation part 102 includes a skeleton estimation part 103, a person identification part 104, a position measurement part 105, and a time measurement part 106.

The skeleton estimation part 103 estimates the skeleton of a person existing in a target space from the RGBD data and generates a skeleton estimation result. The skeleton estimation result includes, for example, three-dimensional positions of a plurality of feature points included in the skeleton. Examples of feature points include joints. The three-dimensional position may be a relative position with respect to a reference point (for example, the position of the depth camera 20). The skeleton estimation part 103 performs skeleton estimation using the depth image alone or using both of the depth image and the RGB image. Since a well-known skeleton estimation technique can be used, a detailed description of skeleton estimation is omitted.

The person identification part 104 receives the skeleton estimation result from the skeleton estimation part 103 and identifies a person existing in a target space on the basis of the skeleton estimation result. The prediction device 100 further includes a skeleton information storage part (not shown) which stores a database in which skeleton information for each subject is recorded and the person identification part 104 refers to the database to identify a person. In the database, skeleton information is associated with the unique ID of the subject. A unique ID corresponds to identification information for identifying a subject. For example, the person identification part 104 calculates a degree of matching between the skeleton estimation result and the skeleton information of each subject and acquires the unique ID associated with the skeleton information with the highest degree of matching. A person other than the target subject may exist in the target space in some cases. When the highest degree of matching is below a predetermined threshold, the person identification part 104 determines that the person existing in the target space is a different person from the subject. Note that the person identification part 104 may identify a person through performing image processing on a color image included in the RGBD data.

The position measurement part 105 receives the skeleton estimation result from the skeleton estimation part 103 and measures the position of the person existing in the target space on the basis of the skeleton estimation result. In one example, the three-dimensional position of the waist of the person is used as the position of the person. The position measurement part 105 calculates the three-dimensional position of the waist from the skeleton estimation result.

The time measurement part 106 measures the time when the RGBD data is acquired. When the RGBD data includes time information, the time measurement part 106 may use the time indicated by the time information as the acquisition time of the RGBD data. Alternatively, the time measurement part 106 may use a time at which the prediction device 100 receives the RGBD data from the depth camera 20 as the acquisition time of the RGBD data.

The position information generation part 102 causes the position information storage part 107 to store a unique ID of the subject specified by the person identification part 104, position information indicating the position measured using the position measurement part 105, and time information indicating the time measured using the time measurement part 106. In the position information storage part 107, the position information is associated with the unique ID and the time information. As described above, the depth camera 20 periodically generates image pairs. Thus, the position information generation part 102 generates position information indicating the positions of the subject at a plurality of consecutive times for each subject.

The degree-of-roaming calculation part 108 calculates a degree of roaming of the subject on the basis of the position information of the subject stored in the position information storage part 107. The degree of roaming indicates the degree of stereotypic behavior relating to walking. The degree of roaming is evaluated using the position information of the subject in a first time period having a predetermined time length T₁. The degree of roaming can be expressed, for example, by the following Expression (1):

$$\begin{matrix}[ Math . 1 ] & \\ W = \frac{D}{{\sum }_{k} {S}_{k}} & ( 1 )\end{matrix}$$

Here, W is the degree of roaming, D is a distance traveled by the subject during a first time period, and Sₖ is a standard deviation of the positions belonging to a cluster k obtained by clustering the positions of the subjects in the first time period. The standard deviation Sₖ is an example of an index representing a size of the cluster k. The denominator of the foregoing Expression (1) corresponds to a range of movement of the subject during the first time period. According to the foregoing Expression (1), the degree of roaming is defined as the distance traveled by the subject during the first time period divided by the range of travel of the subject during the first time period. In this way, the degree of roaming is defined so that the value becomes high when the subject repeatedly moves in a narrow range, that is, when the subject exhibits a stereotypic behavior relating to walking.

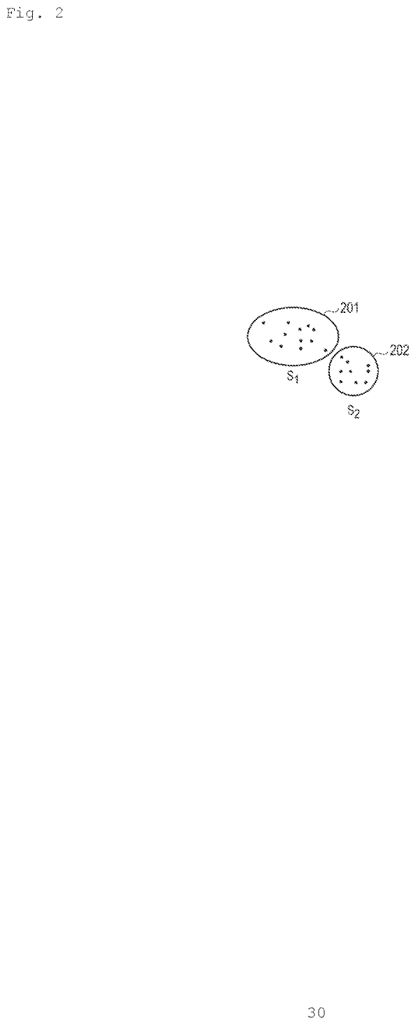

FIG. 2 schematically shows the results of clustering a plurality of locations. As a clustering method, for example, the x-means method can be used. The x-means method determines the number of clusters in accordance with data to be clustered and classifies the data into the determined number of clusters. In the example shown in FIG. 2, the locations are classified into two clusters 201 and 202. A standard deviation S₁ is calculated from the positions belonging to the cluster 201 and a standard deviation S₂ is calculated from the positions belonging to the cluster 202.

Referring to FIG. 1 again, the degree-of-roaming calculation part 108 acquires the position information of the subject during the period from time t-T₁ to time t from the position information storage part 107. The degree-of-roaming calculation part 108 calculates a movement distance D of the subject during the period from time t-T₁ to time t from the acquired position information. The degree-of-roaming calculation part 108 clusters the positions indicated by the acquired position information and calculates the standard deviation Sₖ of the positions belonging to each cluster k. The degree-of-roaming calculation part 108 obtains the degree of roaming W by dividing the movement distance D by the total standard deviation Sₖ, as shown in the foregoing Expression (1).

The degree-of-roaming calculation part 108 stores the calculated degree of roaming in the degree-of-roaming storage part 109. In the degree-of-roaming storage part 109, the degree of roaming is associated with the unique ID of the subject and time information. The time information may be information indicating the time t. The degree-of-roaming calculation part 108 calculates the degree of roaming of the subject at predetermined time intervals T₀ (for example, at intervals of one minute). Thus, the time-series data indicating the degree of roaming at a plurality of consecutive times is stored in the degree-of-roaming storage part 109 for each subject.

The wandering occurrence prediction part 110 predicts the occurrence of wandering in the subject on the basis of the degree of roaming of the subject. For example, the wandering occurrence prediction part 110 calculates the probability of occurrence of wandering of the subject on the basis of the degree of roaming of the subject. The probability of wandering occurrence represents the probability (possibility) of wandering occurring in the subject. The wandering occurrence prediction part 110 determines that wandering occurs when the probability of wandering occurrence exceeds a threshold value and determines that wandering does not occur when the probability of wandering occurrence is the threshold value or less.

In one example, the wandering occurrence prediction part 110 uses one degree of wandering (for example, the latest degree of wandering at the time of prediction) as it is as the probability of wandering occurrence. In another example, the wandering occurrence prediction part 110 calculates the probability of wandering occurrence on the basis of time-series data including a plurality of degrees of roaming in a second time period having a predetermined time length T₂. For example, the wandering occurrence prediction part 110 may use a machine learning model such as a recurrent neural network (RNN). The machine learning model is configured to receive the time-series data of the degree of roaming as an input and output the probability of occurrence of wandering. The model is pre-learned using learning data. The learning data is prepared by actually observing a plurality of people with dementia. The learning data includes time-series data of the degree of roaming immediately before the occurrence of wandering and time-series data of the degree of roaming during normal times. For example, time-series data of the degree of roaming immediately before the occurrence of wandering includes a plurality of degrees of roaming in the time period from time t₀-T₁ to time t₀. Here, the time to is the time a predetermined time (for example, 30 minutes) before the time when the wandering actually occurs. It is possible to generate a model in which the probability of wandering occurring after a predetermined time (for example, 30 minutes) is output by using such learning data for learning.

The notification part 111 notifies the caregiver set for the subject in response to the prediction by the wandering occurrence prediction part 110 that wandering is likely to occur in the subject. For example, the notification part 111 transmits a notification to the communication terminal (for example, the smartphone 25) used by the caregiver indicating that there is a sign of the occurrence of wandering. The smartphone 25 may be configured to display messages via push notifications.

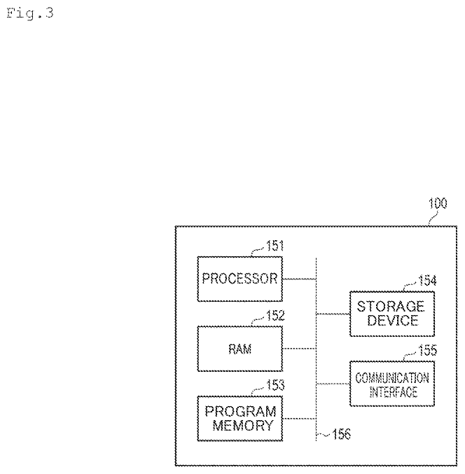

FIG. 3 schematically shows a hardware configuration example of the prediction device 100. As shown in FIG. 3, the prediction device 100 includes a processor 151, a random access memory (RAM) 152, a program memory 153, a storage device 154, a communication interface 155, and a bus 156 as hardware constituent elements. The processor 151 is communicatively connected via the bus 156 with the RAM 152, the program memory 153, the storage device 154, and the communication interface 155.

The processor 151 includes a general-purpose circuit such as a central processing unit (CPU). The RAM 152 includes a volatile memory such as an SDRAM. The RAM 152 is used by the processor 151 as a working memory. The program memory 153 stores programs executed by the processor 151 including detection programs. The program includes computer-executable instructions. For example, a ROM is used as the program memory 153. A partial region of the storage device 154 may be used as the program memory 153.

The processor 151 expands the program stored in the program memory 153 to the RAM 152 and interprets and executes the program. The detection program, when executed using the processor 151, causes the processor 151 to perform a series of processes described herein. In other words, the processor 151 is configured to operate as the acquisition part 101, the position information generation part 102, the degree-of-roaming calculation part 108, the wandering occurrence prediction part 110, and the notification part 111 in accordance with the detection program.

The program may be provided to the prediction device 100 while being stored in a computer-readable recording medium. In this case, the prediction device 100 has a drive which reads data from the recording medium and acquires the program from the recording medium. Examples of recording media include magnetic disks, optical disks (CD-ROM, CD-R, DVD-ROM, DVD-R, and the like), magneto-optical disks (MO and the like), and semiconductor memories. Also, the program may be distributed through a network. Specifically, the program may be stored in a server on a network and the prediction device 100 may download the program from the server.

The storage device 154 includes a non-volatile memory such as a hard disk drive (HDD) or a solid state drive (SSD). The storage device 154 stores the data such as skeleton information, databases, position information, a degree of roaming, and machine learning models described above. The storage device 154 implements the position information storage part 107 and the degree-of-roaming storage part 109.

The communication interface 155 is an interface for communicating with external devices such as the depth camera 20 and the smartphone 25. The communication interface 155 includes wired and/or wireless modules.

Note that the processor 151 may include a dedicated circuit such as an application specific integrated circuit (ASIC) or a field programmable gate array (FPGA), instead of or in addition to the general-purpose circuit. In other words, a part or all of the series of processes described herein may be implemented using a dedicated circuit.

### [Operations]

Operations of the prediction device 100 will be described below.

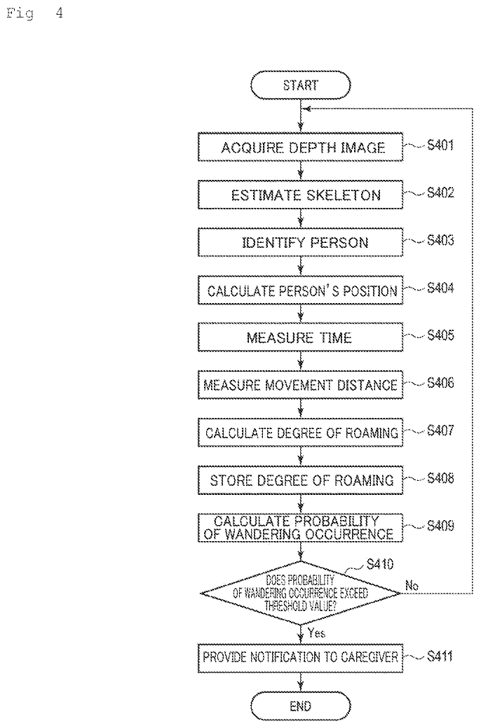

FIG. 4 schematically shows an example of a prediction method performed by the prediction device 100. Here, for the sake of simplicity, a case in which there are one depth camera and one subject will be described. When a plurality of depth cameras are provided, the processes shown in Steps S401 to S405 in FIG. 4 are performed for each depth camera. When dealing with a plurality of subjects, the processes shown in Steps S406 to S411 in FIG. 4 are performed for each subject.

In Step S401, the acquisition part 101 acquires RGBD data from the depth camera 20. The RGBD data includes a depth image in a target space and a color image in a target space.

In Step S402, the skeleton estimation part 103 estimates the skeleton of a person existing in the target space on the basis of the RGBD data and generates skeleton estimation results including the three-dimensional positions of feature points in the skeleton.

In Step S403, the person identification part 104 identifies a person existing in the target space on the basis of the skeleton estimation result obtained using the skeleton estimation part 103. For example, the person identification part 104 determines whether a person existing in the target space is the subject.

In Step S404, the position measurement part 105 measures the three-dimensional position of the person existing in the target space on the basis of the skeleton estimation result obtained using the skeleton estimation part 103.

In Step S405, the time measurement part 106 measures the time at which the RGBD data is acquired. For example, the time measurement part 106 obtains the time indicated by the time information included in the RGBD data as the data acquisition time.

When the person identification part 104 determines that the person existing in the target space is the subject in Step S403, the unique ID of the subject, the position measured in Step S404, and the time measured in Step S405 are stored in the position information storage part 107 in association with each other.

A series of processes shown in Steps S401 to S405 are performed each time the depth camera 20 generates RGBD data. Thus, the time-series data indicating the position of the subject is recorded.

In Step S406, the degree-of-roaming calculation part 108 calculates the distance traveled by the subject in the first time period on the basis of the position information of the subject stored in the position information storage part 107. Assuming that the reference time (for example, the current time) is t₁, the first time period is the period from time t₁-T₁ to time t₁. The degree-of-roaming calculation part 108 calculates the movement distance of the subject in the first time period from the position information of the subject in the first time period.

In Step S407, the degree-of-roaming calculation part 108 calculates the degree of roaming of the subject in the first time period. For example, the degree-of-roaming calculation part 108 clusters the positions indicated by the position information of the subject in the first time period and calculates the standard deviation of the positions belonging to each cluster. The degree-of-roaming calculation part 108 calculates the degree of roaming on the basis of the movement distance calculated in Step S406 and the standard deviation of each cluster. For example, as shown in the foregoing Expression (1), the degree-of-roaming calculation part 108 calculates the sum of the standard deviations of the clusters as the movement range of the subject in the first time period and calculates the degree-of-roaming by dividing the movement distance in the first time period by the movement range of the subject in the first time period.

In Step S408, the degree-of-roaming calculation part 108 stores the calculated degree-of-roaming in the degree-of-roaming storage part 109 in association with the time t and the unique ID of the subject.

A series of processes shown in Steps S406 to S408 are performed at predetermined time intervals T₀. Thus, the time-series data indicating the degree-of-roaming of the subject is recorded.

In Step S409, the wandering occurrence prediction part 110 calculates the subject's probability of wandering occurrence on the basis of the subject's degree-of-roaming stored in the degree-of-roaming storage part 109. For example, the wandering occurrence prediction part 110 acquires time-series data including a plurality of degrees of roaming in the second time period from the degree-of-roaming storage part 109 and calculates the subject's probability of wandering occurrence on the basis of this time-series data. Assuming that the reference time (for example, the current time) is t₂, the second time period is, for example, the period from time t₂-T₂ to time t₂. The wandering occurrence prediction part 110 inputs time-series data including a plurality of degrees of roaming in the second time period to the machine learning model and obtains the probability of wandering occurrence output from the machine learning model as the subject's probability of wandering occurrence.

In Step S410, the wandering occurrence prediction part 110 determines whether the probability of wandering occurrence obtained in Step S409 exceeds a predetermined threshold value. When the probability of wandering occurrence is the threshold value or less (Step S410; No), the wandering occurrence prediction part 110 determines that the probability of wandering occurrence in the subject is low and the process returns to Step S401.

When the probability of wandering occurrence exceeds the threshold value (Step S410; Yes), the wandering occurrence prediction part 110 determines that there is a high probability that wandering will occur in the subject and the process proceeds to Step S411. In Step S411, the notification part 111 outputs a notification indicating that the subject will wander. For example, the notification part 111 transmits the notification to the smartphone 25 used by a caregiver set for the subject.

A series of processes shown in Steps S409 to S411 are performed at time intervals equal to or longer than the time interval T₀.

### Effects

The prediction device 100 calculates a degree of roaming indicating a degree of stereotypic behavior relating to walking for the subject on the basis of the position information of the subject, predicts the occurrence of psycho-behavioral symptoms in the subject on the basis of the degree of roaming of the subject, and outputs a notification in response to prediction that psycho-behavioral symptoms will occur in the subject. The stereotypic behaviors relating to walking are quantified. For example, the degree of roaming may be defined as the distance traveled by the subject during the first time period divided by the range of travel of the subject during the first time period. For example, the prediction device 100 may cluster the positions of the subject in the first time period, calculate the size of at least one cluster obtained by clustering, and calculate the range of motion of the subject in the first time period on the basis of the size of at least one cluster. It becomes possible to predict the occurrence of wandering in advance by quantifying stereotypic behavior relating to walking.

The prediction device 100 may receive as input time-series data of the degree of roaming and make predictions using a learned model configured to output the probability of the occurrence of wandering. The prediction device 100 inputs time-series data including a plurality of degrees of roaming of the subject in the second time period into the learned model and probabilities output from the trained model may be obtained and the occurrence of wandering in the subject may be predicted on the basis of a comparison of the probability output from the trained model to a predetermined threshold value. Prediction can be performed with higher accuracy than rule-based prediction.

The prediction device 100 measures the position of the subject on the basis of the depth image and the color image of the target space in which the subject exists. Thus, it is possible to capture the fine movements of the subject and highly accurate position measurement is possible. For example, an indoor space such as a room is assumed as the target space. When the target space is an indoor space, the accuracy of positioning using a global positioning system (GPS) system decreases. Here, the accuracy of position measurement using depth images does not decrease.

### [Modifications]

In the above-described embodiment, the occurrence of wandering is predicted on the basis of the degree of roaming. Prediction based on the degree of roaming can also be applied to symptoms (abnormal behavior) other than wandering.

In the embodiments described above, the depth camera 20 generates depth images and color images. In other embodiments, the depth camera 20 may generate depth images without generating color images. In the embodiments in which depth camera 20 generates only depth images, the position information generation part 102 generates position information for the subject on the basis of the depth images.

In the embodiments described above, the depth camera 20 is used for obtaining the position information of the subject. Any device which can obtain the position information of the subject may be used.

Note that the present invention is not limited to the above-described embodiments and can be variously modified in the implementation stage without departing from the gist of the present invention. Furthermore, each embodiment may be implemented in combination as appropriate, in which case combined effects can be obtained. Furthermore, various inventions are included in the above embodiments and various inventions can be extracted by combinations selected from the disclosed plurality of constituent elements. For example, even if some constituent elements are deleted from all the constituent elements shown in the embodiment, if the problem can be solved and effects can be obtained, the configuration in which these constituent elements are deleted can be extracted as an invention.

### REFERENCE SIGNS LIST

10 Prediction system
20 Depth camera
25 Smartphone
100 Prediction device
101 Acquisition part
102 Position information generation part
103 Skeleton estimation part
104 Person identification part
105 Position measurement part
106 Time measurement part
107 Position information storage part
108 Degree-of-roaming calculation part
109 Degree-of-roaming storage part
110 Wandering occurrence prediction part
111 Notification part
151 Processor
152 RAM
153 Program memory
154 Storage device
155 Communication interface
156 Bus

The invention claimed is:

1. A prediction device, comprising:
a position information generation part, including one or more processors, configured to generate position information of a subject;
a degree-of-roaming calculation part, including one or more processors, configured to calculate a degree of roaming indicating a degree of stereotypic behavior relating to walking for the subject on the basis of the position information of the subject;
a prediction part, including one or more processors, configured to predict occurrence of a predetermined symptom in the subject on the basis of the degree of roaming of the subject; and
a notification part, including one or more processors, configured to output a notification in response to prediction that symptom will occur in the subject.

2. The prediction device according to claim 1, wherein the degree-of-roaming calculation part is configured to calculate a movement distance of the subject in a first time period on the basis of the position information of the subject, calculate a movement range of the subject in the first time period on the basis of the position information of the subject, and calculate a degree of roaming by dividing the movement distance of the subject in the first time period by the movement range of the subject in the first time period.

3. The prediction device according to claim 2, wherein the degree-of-roaming calculation part is configured to cluster the positions of the subject in the first time period indicated by the position information of the subject, calculate a size of at least one cluster obtained by clustering, and calculate a movement range of the subject during the first time period on the basis of the size of the at least one cluster.

4. The prediction device according to claim 1, further comprising:
a model configured to receive, as an input, the time series data of the degree of roaming and to output the probability of occurrence of the symptom,
wherein the prediction part is configured to input time-series data including a plurality of degrees of roaming of the subject in a second time period into the model, obtain the probability output from the model, and predict occurrence of the symptom in the subject on the basis of a comparison between the probability output from the model and a predetermined threshold value.

5. The prediction device according to claim 1, wherein the position information generation part is configured to acquire a depth image indicating depth information of a target space in which the subject exists, measure a position of the subject on the basis of the depth image, and generate the position information of the subject.

6. The prediction device according to claim 1, wherein the symptom is wandering.

7. A prediction method, comprising:
generating position information of a subject;
calculating a degree of roaming of the subject indicating a degree of stereotypic behavior relating to walking on the basis of the position information of the subject;
predicting occurrence of a predetermined symptom in the subject on the basis of the degree of roaming of the subject; and
outputting a notification in response to predicting that the symptom will occur in the subject.

8. A non-transitory computer readable storage medium storing a program for causing a computer to performs operations of a prediction method, the operations comprising:
generating position information of a subject;
calculating a degree of roaming of the subject indicating a degree of stereotypic behavior relating to walking on the basis of the position information of the subject;
predicting occurrence of a predetermined symptom in the subject on the basis of the degree of roaming of the subject; and
outputting a notification in response to predicting that the symptom will occur in the subject.

9. The prediction method according to claim 7, further comprising:
calculating a movement distance of the subject in a first time period on the basis of the position information of the subject, calculating a movement range of the subject in the first time period on the basis of the position information of the subject, and calculating a degree of roaming by dividing the movement distance of the subject in the first time period by the movement range of the subject in the first time period.

10. The prediction method according to claim 9, further comprising:
clustering the positions of the subject in the first time period indicated by the position information of the subject, calculating a size of at least one cluster obtained by clustering, and calculating a movement range of the subject during the first time period on the basis of the size of the at least one cluster.

11. The prediction method according to claim 7, further comprising:
inputting time-series data including a plurality of degrees of roaming of the subject in a second time period into a model configured to receive, as an input, the time series data of the degree of roaming and to output the probability of occurrence of the symptom, obtaining the probability output from the model, and predicting occurrence of the symptom in the subject on the basis of a comparison between the probability output from the model and a predetermined threshold value.

12. The prediction method according to claim 7, further comprising:
acquiring a depth image indicating depth information of a target space in which the subject exists, measuring a position of the subject on the basis of the depth image, and generating the position information of the subject.

13. The prediction method according to claim 7, wherein the symptom is wandering.

14. The non-transitory computer readable storage medium according to claim 8, the operations further comprising:
calculating a movement distance of the subject in a first time period on the basis of the position information of the subject, calculating a movement range of the subject in the first time period on the basis of the position information of the subject, and calculating a degree of roaming by dividing the movement distance of the subject in the first time period by the movement range of the subject in the first time period.

15. The non-transitory computer readable storage medium according to claim 11, the operations further comprising:
clustering the positions of the subject in the first time period indicated by the position information of the subject, calculating a size of at least one cluster obtained by clustering, and calculating a movement range of the subject during the first time period on the basis of the size of the at least one cluster.

16. The non-transitory computer readable storage medium according to claim 8, the operations further comprising:
inputting time-series data including a plurality of degrees of roaming of the subject in a second time period into a model configured to receive, as an input, the time series data of the degree of roaming and to output the probability of occurrence of the symptom, obtaining the probability output from the model, and predicting occurrence of the symptom in the subject on the basis of a comparison between the probability output from the model and a predetermined threshold value.

17. The non-transitory computer readable storage medium according to claim 8, the operations further comprising:
acquiring a depth image indicating depth information of a target space in which the subject exists, measuring a position of the subject on the basis of the depth image, and generating the position information of the subject.

18. The non-transitory computer readable storage medium according to claim 8, wherein the symptom is wandering.
